(19) United States
(12) Patent Application Publication
(10) Pub. No.: US 2016/0235608 A1
(43) Pub. Date: Aug. 18, 2016

(54) THERMOPLASTIC ARTICLE WITH ODOR CONTROL SYSTEM

(71) Applicant: KIMBERLY-CLARK WORLDWIDE, INC., Neenah, WI (US)

(72) Inventors: Shiming Zhuang, Menasha, WI (US); Peiguang Zhou, Appleton, WI (US); John Gavin MacDonald, Decatur, GA (US); Rebecca Ann Vongsa, Neenah, WI (US); Andrew Mark Long, Appleton, WI (US); David William Koenig, Menasha, WI (US)

(73) Assignee: Kimberly-Clark Worldwide, Inc., Neenah, WI (US)

(21) Appl. No.: 15/025,435

(22) PCT Filed: Sep. 29, 2014
(86) PCT No.: PCT/US14/57967

§ 371 Date: Mar. 28, 2016

Related U.S. Application Data
(60) Provisional application No. 61/909,256, filed on Nov. 26, 2013.
(60) Provisional application No. 61/884,574, filed on Sep. 30, 2013.

(51) Int. Cl. A61F 13/84; A61L 15/20; A61L 15/46; A61L 15/18; C08J 5/18; A61L 15/22

(52) U.S. Cl. CPC A61F 13/8405; C08J 5/18; A61L 15/225; A61L 15/46; A61L 15/18; A61L 15/20; C08J 2329/04; A61F 2013/8414; A61F 2013/8408; A61F 2013/8423; A61F 2013/8435; B29C 47/065

(57) ABSTRACT

Disclosed is an article for urine odor control. The article may be in the form of a film The article is a homogeneous material may include a water-soluble polymer having an extrusion temperature of 50 to 150 C, and between 0.1% to 50% by weight of an odor-control system. The odor control system may include one or more the following: an enzyme inhibitor, an odor absorber, a blocking agent or an antimicrobial agent. The article may be placed in an absorbent member, wherein the absorbent member is a bandage, a medical drape, a wipe, a towel, a sheet, a pad, a pant or a diaper.

This application claims priority as a continuation of Application No. 61/884574, filed on Sep. 30, 2013; and Application No. 61/909256 filed on Nov. 26, 2013. The entirety of Application No. 61/884574 and Application No. 61/909256 is incorporated herein by reference.

### BACKGROUND

Disposable absorbent products such as pads, pants, diapers and shields have been used for many years by a wide variety of people. For instance, women may use pads during their menstrual period, and those suffering from incontinence may use a pad, pant or shield depending on their needs. Children of course wear diapers prior to potty training. In all such uses, adequate odor control is a significant desire to preserve the dignity of the wearer, especially those who are incontinent.

All disposable absorbent products have a means to absorb and contain wetness. One common way of achieving this is to incorporate an absorbent member into the article. The absorbent member typically includes a cellulosic pulp and a superabsorbent material combined into a core, with a non-woven liner material covering the core.

Known odor control methods include surface-treating various components of the absorbent article. One way to treat odor is to apply citric acid to the cellulosic pulp located in the core (e.g. GOODNITE pants made by Kimberly-Clark Corporation use this technique). The citric acid neutralizes odorants to remove odors. More specifically, the citric acid's low pH reduces odor-producing bacteria and neutralizes ammonia and amines.

Another way to treat odor is to print activated carbon onto the liner material covering the core (e.g. some POISE pads made by Kimberly-Clark Corporation contain activated carbon, see for instance U.S. Pat. No. 8,287,510, issued to MacDonald et al.). The activated carbon absorbs odors.

Yet another way to treat odor is to apply a fragrance to the liner material covering the core. This serves to mask the odor. All such approaches are based on surface treatment and have demonstrated some success. However, there is a desire for improvement, especially in the area of urine-odor control.

Odor control is challenging in any of the above-noted techniques, mainly due to poor stability of the active, the inability to deliver multiple actives, and the inability to deliver enough actives to fully treat the odor. What is needed is a cost-effective method and composition or device to treat odor. Desirably, the method does not add many additional steps to the manufacturing process. Further, it is desirable that the method treat odor using more than one chemical approach.

### SUMMARY

One aspect of the present disclosure is an extruded water-soluble article made using a homogeneous material comprising a water-soluble, amorphous polyvinyl alcohol matrix having an extrusion temperature of 90° C. to 125° C. The article has between 0.1% to 50% by weight of an odor-control system incorporated therein. The odor-control system is selected from the group consisting of an enzyme inhibiter, a blocking agent, an antimicrobial agent, an odor-absorption agent, or a combination thereof.

Another aspect of the present disclosure is a personal absorbent article that has the following components: an absorbent member disposed between a water-impermeable backsheet and a water-permeable liner, wherein the liner has a body-facing surface and an opposite garment-facing surface; and the extruded water-soluble article attached to the liner or a surface of the absorbent member.

Yet another aspect of the present disclosure is an extruded water-soluble article made with a homogeneous material comprising a water-soluble, polymer having an extrusion temperature of 50 to 150° C. Between 0.1% to 50% by weight of an odor-control system comprising silver-based zeolite and polyoxyethylene glyceryl monococoate.

Other features and aspects of the invention will become apparent by consideration of the detailed description and accompanying drawings.

### BRIEF DESCRIPTION OF THE DRAWINGS

The foregoing and other features and aspects of the present invention and the manner of attaining them will become more apparent, and the invention itself will be better understood by reference to the following description, appended claims and accompanying drawings, where;

FIG. 1 is a chart showing the dissolution time for one embodiment of a film according to the present disclosure;

FIGS. 2-4 are charts showing zone of inhibition on films containing various biocides of the present disclosure;

FIG. 5 is a chart showing the stress and strain properties of films having varying percentages of a first embodiment of an antimicrobial agent;

FIG. 6 is a chart showing the modulus and toughness of the films of FIG. 5;

FIG. 7 is a chart showing the stress and strain properties of films having varying percentages of a second embodiment of an antimicrobial agent;

FIG. 8 is a chart showing the modulus and toughness of the films of FIG. 7;

FIG. 9 is a chart showing the stress and strain properties of films having varying percentages of a third embodiment of an antimicrobial agent;

FIG. 10 is a chart showing the modulus and toughness of the films of FIG. 9;

FIG. 11 is a side elevation of one embodiment of a laminate according to the present disclosure;

FIG. 12 is an exploded side elevation of one embodiment of a personal absorbent article;

FIG. 13 is a side cross-sectional view of one embodiment of an absorbent article according to the present disclosure;

FIG. 14 is a schematic showing various steps of a zone of inhibition test according to the disclosure;

FIG. 15 is a schematic showing how test material is spread onto a medium in the test of FIG. 14;

FIG. 16 is a chart showing the dissolution time of various films having an odor control system of the present disclosure;

FIG. 17 is a chart showing the tensile stress of various films having an odor control system of the present disclosure;

FIG. 18 is a chart showing the tensile strain (elongation) of various films having an odor control system of the present disclosure;

FIG. 19 is a schematic cross-section of an absorbent article showing the various positions a film of the present disclosure may be disposed;

FIG. 20 is a chart showing the fluid intake time for various films having an odor control system of the present disclosure disposed at various positions in the article of FIG. 19;

FIG. 21 is a chart showing the fluid flowback of various films having an odor control system of the present disclosure disposed at various positions in the article of FIG. 19;

FIG. 22 is a chart showing the diffusion distance and the leftover ratio of various films having an odor control system of the present disclosure, disposed at various positions in the article of FIG. 19; and

FIGS. 23 and 24 are charts showing the results of Odor Ranking Panel (ORP) Tests for various films having an odor control system of the present disclosure.

### DETAILED DESCRIPTION

It is to be understood by one of ordinary skill in the art that the present disclosure is a description of exemplary aspects of the present invention only, and is not intended as limiting the broader aspects of the present invention.

The term “laminate” refers to a material where a film structure is adhesively or non-adhesively bonded to a web such as a nonwoven or tissue material.

The term “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. In the particular case of a coform process, the meltblown fiber stream intersects with one or more material streams that are introduced from a different direction. Thereafter, the meltblown fibers and other optional materials are carried by the high velocity gas stream and are deposited on a collecting surface. The distribution and orientation of the meltblown fibers within the formed web is dependent on the geometry and process conditions. Exemplary meltblown processes are described in various patents and publications, including NRL Report 4364, “Manufacture of Super-Fine Organic Fibers” by V. A. Wendt, E. L. Boone and C. D. Fluharty; NRL Report 5265, “An Improved Device For the Formation of Super-Fine Thermoplastic Fibers” by K. D. Lawrence, R. T. Lukas and J. A. Young; and U.S. Pat. No. 3,849,241 to Butin et al. and U.S. Pat. No. 5,350,624 to Georger et al., each of which is incorporated herein by reference in a manner that is consistent herewith.

The terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblown processes, spunbond processes, air laying processes, wet layering processes and bonded-carded-web processes.

The term “personal care absorbent articles” or “absorbent articles” in the context of this disclosure includes, but is not limited to diapers, diaper pants, training pants, absorbent underpants, incontinence products, and urinary shields; and the like.

The terms “spunbond” and “spunbond fiber” refer to fibers which are formed by extruding filaments of molten thermoplastic material from a plurality of fine, usually circular, capillaries of a spinneret, and then rapidly reducing the diameter of the extruded filaments.

The term “% by weight,” “weight %,” “wt %” or derivative thereof, when used herein, is to be interpreted as based on the dry weight, unless otherwise specified.

These terms may be defined with additional language in the remaining portions of the specification.

The present disclosure is generally directed to an extruded, water-soluble, thermoplastic article into which an active agent has been incorporated. The thermoplastic water-soluble, polymer from which the article is made has an extrusion temperature of 90° C. to 150° C. The combination of the polymer and active agent(s) is a homogeneous blend having an extrusion temperature of 50° C. to 125° C. The articles made from the homogeneous blend include films, fibers, pellets, or other extruded shapes. The articles may be used to control microbes, odor, or both.

### Materials

The materials from which the water-soluble, thermoplastic material of the present disclosure is made generally include a polymer and one or more active agents. Other optional materials that improve the performance, look, feel and/or durability may be added to the thermoplastic material.

Polymer: Generally, the polymer used in the present disclosure is polyvinyl alcohol (PVOH), polyethylene oxide (PEO), polyethylene glycol (PEG), polyacylate (acid), polyacylamide, polyester, or a combination of one or more of these polymers. Suitable polymers have an extrusion temperature of 90° C. to 150° C.

One desirable polymer is a highly amorphous vinyl alcohol polymer, sold as “NICHIGO G-POLYMER,” available from Soarus L.L.C., Arlington Heights, Illinois. This particular polymer has a molecular weight of 10,000 to 50,000, and a relatively low crystallinity of 5 to 25%.

In one aspect, a copolymer such as ethylene vinyl acetate (EVA) may be combined with the base polymer. It is contemplated that the article of the present disclosure may include up to 30% by weight EVA. EVA aids in extrusion process, provides a means to control the water dissolution speed, and lowers the overall cost if the extruded material.

Active Agent: In one aspect, the active agent is made from one or more antimicrobial agents. Suitable antimicrobials include biocides such as benzalkonium chloride (“BAC”), didecyl dimethyl ammonium chloride (“DDAC”), and zeolite, which contains silver (“CWT-A”). Other possible active agents include: isothiazolone, alkyl dimethyl ammonium chloride, a triazine, 2-thiocyanomethylthio benzothiazol, methylene bis thiocyanate, acrolein, dodecylguanidine hydrochloride, a chlorophenol, a quaternary ammonium salt, gluteraldehyde, a dithiocarbamate, 2-mercatobenzothiazole, para-chloro-meta-xylenol, silver, chlorohexidine, polyhexamethylene biguanide, a n-halamine, triclosan, a phospholipid, an alpha hydroxyl acid, 2,2-dibromo-3-nitrilopropionamide, 2-bromo-2-nitro-1,3-propanediol, farnesol, iodine, bromine, hydrogen peroxide, chlorine dioxide, a botanical oil, a botanical extract, chlorine, sodium hypochlorite, or combinations thereof.

The amount of active agent that is loaded into an article is limited due to the integrity of the resulting article structure. If there is too much active agent in an article, it may be unduly weakened. In one aspect, the sum of the active agent(s) is present in a total amount of 0.1% to 50% by weight of the article, or a total amount of 1% to 20% by weight of the article. In another aspect, the sum of the active agent(s) is present in a total amount of 2% to 10% by weight of the article.

Optional Materials: Besides the components noted above, still other additives may also be incorporated into the composition, such as fragrances, melt stabilizers, dispersion aids (e.g., surfactants), processing stabilizers, heat stabilizers, light stabilizers, UV stabilizers, antioxidants, heat aging stabilizers, whitening agents, antiblocking agents, antistatic agents, bonding agents, lubricants, colorants, etc.

In one aspect of the present disclosure, the extruded water-soluble article includes up to 50% thermoplastic starch by weight. The starch acts as a filler to reduce the overall cost of the extruded article. The extruded article may contain as much as 30% starch. One desirable water-soluble thermal starch is a cellulose-based starch obtained from various plant sources, hemicelluloses, modified cellulose (hydroxylalkyl cellulose, cellulose ethers, cellulose esters, etc.), and the like. When a starch is employed, the amount of such additional material may range from about 0.1 wt. % to about 50 wt. % of the homogeneous blend, in some embodiments from about 0.5 wt. % to about 40 wt. %, and in some embodiments, from about 1 wt. % to about 30 wt. %.

Dispersion aids may also be employed to help create a uniform dispersion of the active agent/plasticizer. When employed, the dispersion aid(s) typically constitute from about 0.01 wt. % to about 10 wt. % of the homogeneous blend, in some embodiments from about 0.1 wt. % to about 5 wt. %, and in some embodiments, from about 0.5 wt. % to about 4 wt. %.

The composition may also contain a preservative or preservative system to inhibit the growth of microorganisms over an extended period of time. Suitable preservatives may include, for instance, alkanols, disodium EDTA (ethylenediamine tetraacetate), EDTA salts, EDTA fatty acid conjugates, isothiazolinone, benzoic esters (parabens) (e.g., methylparaben, propylparaben, butylparaben, ethylparaben, isopropylparaben, isobutylparaben, benzylparaben, sodium methylparaben, and sodium propylparaben), benzoic acid, propylene glycols, sorbates, urea derivatives (e.g., diazolindinyl urea), and so forth. Other suitable preservatives include those sold by Sutton Labs, such as “Germall 115” (amidazoiidinyl urea), “Germall II” (diazolidinyl urea), and “Germall Plus” (diazolidinyl urea and iodopropynyl butylcarbonate). Another suitable preservative is Kathon CG.RTM., which is a mixture of methylchloroisothiazolinone and methylisothiazoiinone available from Rohm & Haas; Mackstat H 66 (available from McIntyre Group, Chicago, Ill.). Still another suitable preservative system is a combination of 56% propylene glycol, 30% diazolidinyl urea, 11% methylparaben, and 3% propylparaben available under the name GERMABEN.RTM. H from International Specialty Products of Wayne, N.J.

To better enhance the benefits to consumers, other optional ingredients may also be used. For instance, some classes of ingredients that may be used include, but are not limited to:

antioxidants (for product integrity); astringents-cosmetic (for inducing a tingling sensation on skin); colorants (for imparting color to the product); deodorants (for reducing or eliminate unpleasant odor and protect against the formation of malodor on body surfaces); fragrances (for consumer appeal); skin conditioning agents; and skin protectants (a drug product which protects injured or exposed skin or mucous membrane surface from harmful or annoying stimuli).

### Method of Manufacture

In one aspect of the disclosure, a method of making an extruded article may include the following steps. First, a homogenous blend is formed by combining the polymer with at least one active ingredient and possibly, one or more of the optional ingredients described herein. In one desired embodiment, the polymer is an amorphous, water-soluble vinyl alcohol as described herein. Second, the homogeneous blend is extruded to form an article.

The homogeneous blend has an extrusion temperature of 50° C. to 125° C., or possibly, 90° C. to 125° C. This low extrusion-temperature profile is desirable because some active agents of interest have poor thermal stability. By using a low extrusion-temperature, a wider variety of active agents may be incorporated into the homogenous blend.

Exemplary manufacturing equipment, a method of making articles, and exemplary articles are described herein.

Extrusion Method: The composition of the present disclosure is formed by processing the components together in a melt-blending device (e.g., extruder). The mechanical shear and heat provided by the device allows the components to be blended together in a highly efficient manner without the use of a solvent. Batch and/or continuous melt blending techniques may be employed in the present disclosure. For example, a mixer/kneader, Banbury mixer, Farrel continuous mixer, single-screw extruder, twin-screw extruder, roll mill, etc., may be utilized. One particularly suitable melt-blending device is a twin-screw extruder (e.g., PRISM USALAB x16, available from Thermo Electric Co., Inc., N.J.).

The polymer and the active agent(s), along with any optional ingredients, form a homogeneous blend. For example, the materials may be blended at a shear/pressure and temperature sufficient to ensure adequate mixing (e.g., at or above the softening point of the polymer), but without adversely impacting the physical properties of the active agent. For example, melt-blending typically occurs at a temperature of from about 50° C. to about 150° C., in some embodiments, from about 90° C. to about 130° C., and in some embodiments from about 110° C. to about 125° C. These lower processing temperatures prevent degradation of the active agent.

Once formed, the homogeneous blend of the present disclosure may be used to create a variety of forms, such as films, fibers, rods, bars or other shapes.

Films: In one particular embodiment, the homogeneous blend is formed into a film, either alone or in conjunction with an additional film-forming material. The film may be used in a wide variety of applications, such as a carrier of active agents for medical products, garments, absorbent articles, etc. The film may have a mono- or multi-layer configuration. Any known technique may be used to form a film from the compounded material such as extrusion coating, coextrusion of the layers, or any conventional layering process.

The process to make the antimicrobial reservoir film is relatively fast considering the high amounts of active agent that can be added to the extrusion process. In one particular embodiment, the film may be formed by flat die extrusion technique. Processes for producing such extrusions are described, for instance, in U.S. Pat. No. 7,666,337 to Yang et al.; U.S. Pat. No. 5,091,228 to Fuji et al; and U.S. Pat. No. 4,136,145 to Fuchs et al.; all of which are incorporated herein in their entirety by reference thereto for all purposes.

In yet another embodiment, however, the film is formed using a casting or blowing technique.

Regardless of how the film is formed, it may be optionally oriented in one or more directions to further improve film uniformity and reduce thickness. For example, the film may be immediately reheated to a temperature below the melting point of one or more polymers in the film, but high enough to enable the composition to be drawn or stretched. In the case of sequential orientation, the “softened” film is drawn by rolls rotating at different speeds or rates of rotation such that the sheet is stretched to the desired draw ratio in the longitudinal direction (machine direction). The film may be made into thicknesses ranging from 0.01 mm up to about 1 mm, or in other aspects, from 0.05 mm to 0.20 mm.

The multi-layer film may contain from two (2) to nine (9) layers, and in some embodiments, from three (3) to five (5) layers. In one example, the multi-layer film has one base layer and one skin layer. The base layer and/or skin layer may contain the active agent(s). The ratio between the layers may range from 1 to 20.

In another example, there is a three-layered film having a core layer “C” that is contains an active agent as described herein. The outer skin layers “S” may act as a protective layer to the core. The ratio between the layers may range from 2% to 98% of the core layer and from 10% to 90% of the two combined skin layers. For instance, the core layer may be up to about 30%, up to about 40%, up to about 50%, up to about 60%, or up to about 70% of the total thickness of the multi-layer film. Each skin layer may be up to about 15%, or up to about 25%, or up to about 35% of the total thickness of the multi-layer film.

The film, either mono- or multi-layered, may be wound and stored on a take-up roll. Various additional potential processing and/or finishing steps known in the art, such as slitting, treating, aperturing, printing graphics may be performed.

In one aspect, the extruded water-soluble film has a basis weight of 5 gsm to 500 gsm. In another aspect, the water-soluble film has a basis weight of 20 gsm to 200 gsm.

In one aspect, the extruded water-soluble film has a tensile strength of 0.5 MPa to 50 MPa according to the Tensile Test of the present disclosure. In another aspect, the film has a tensile strength of 1 MPa to 25 MPa according to the same test.

In one aspect, the extruded water-soluble film has a water dissolution speed from 5 seconds to 30 minutes as determined by the Water Dissolution Test of the present disclosure. In another aspect, the extruded water-soluble article film has a water dissolution speed of 30 seconds to 5 minutes as determined by the same test.

In one aspect, the extruded water-soluble film has an elongation of 5% to 500% according to the Tensile Test of the present disclosure. In another aspect, the film has an elongation of 10% to 100% according to the same test.

Articles: The homogeneous blend of the present invention may also be used to form other types of articles. In one aspect, the extruded water-soluble article is a rod having a circular- or elliptical-shaped extrusion profile. In another aspect, the extruded water-soluble article is a rod having the geometric extrusion profile of a polygon with three to ten sides (e.g. a triangle to a decagon). The rod may be cut into pellets for later processing.

Referring to FIG. 13, a laminate may be formed by extruding the homogeneous blend 24 onto a carrier substrate 22, forming a bond therebetween. The carrier substrate 22 may be a nonwoven or woven material, or a tissue.

### Applications

Absorbent Articles: The film of the present invention is particularly suitable for use in an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins, pantiliners, etc.), swim wear, baby wipes, and so forth; medical absorbent articles, such as garments, fenestration materials, underpads, bedpads, bandages, absorbent drapes, and medical wipes; food service wipers; clothing articles; and so forth. Several examples of such absorbent articles are described in U.S. Pat. No. 5,649,916 to DiPalma, et al.; U.S. Pat. No. 6,110,158 to Kielpikowski; U.S. Pat. No. 6,663,611 to Blaney, et al., which are incorporated herein in their entirety by reference thereto for all purposes. Still other suitable articles are described in U.S. Patent Application Publication No. 2004/0060112 A1 to Fell et al., as well as U.S. Pat. No. 4,886,512 to Damico et al.; U.S. Pat. No. 5,558,659 to Sherrod et al.; U.S. Pat. No. 6,888,044 to Fell et al.; and U.S. Pat. No. 6,511,465 to Freiburger et al., all of which are incorporated herein in their entirety by reference thereto for all purposes. Materials and processes suitable for forming such absorbent articles are well known to those skilled in the art.

The present invention may be better understood with reference to the examples presented herein.

First Exemplary Absorbent Article: Referring to FIG. 12, in one aspect of the disclosure, a personal absorbent article 30 includes an absorbent member 32 sandwiched between a water-impermeable backsheet 34 and a water-permeable liner 36, wherein liner 36 has a body-facing surface 38 and an opposite outward-facing surface 40. A surge member 31 is located between the liner 63 and absorbent member 32. A film 41 of the present disclosure is attached to either the outward-facing surface 33 of the surge member 31 or desirably, an inner-facing surface 35 of the surge member 31. Desirably, film 41 is in direct contact with surge member 31. Should a multi-layer film be used for film 41, the layer containing the largest amount of active agent is adjacent surge member 31 so that the active agent can more easily leach through the liner to contact the wearer's body.

As described, film 41 is made from materials that include a water-soluble, polymer that may have an extrusion temperature of 90 to 150° C.; a plasticizer; and one or more volatile active agents in a total amount of 0.1% to 50% by weight of the article.

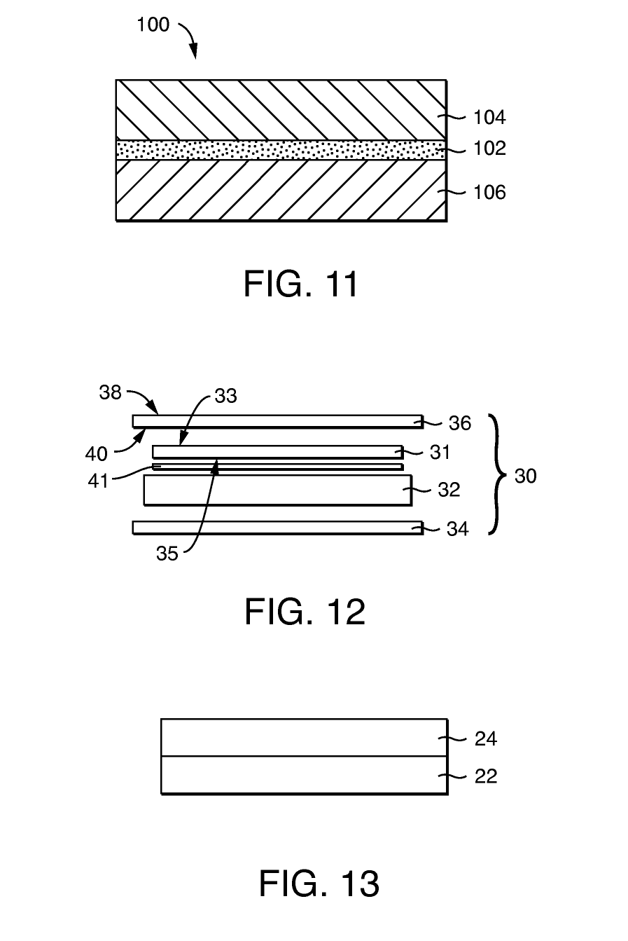

Second Exemplary Absorbent Article: Referring to FIG. 11, in one aspect, the film of the present disclosure is laminated to other layers (e.g., nonwoven or cellulose-fiber based web materials). One particular application of a laminate structure is that of a three-layer wipe 100. In this embodiment, the core layer 102 is a film containing at least the active agent(s) of the present disclosure. Desirably, the outer layers 104 and 106 that surround the core layer are natural or synthetic fiber based web materials (e.g. tissue, paper, spunbond, spunbond-meltblown-spunbond composite, coform, airlaid, etc.).

### Experimental Data—Antimicrobial Activies

Provided is experimental data for three antimicrobial agents that act as biocides, namely, zeolite (silver-aluminosilicate), benzalkonium chloride, and didecyl dimethyl ammonium chloride. Tests were performed on the various codes for each biocide to determine dissolution, zone of inhibition, and mechanical properties.

TABLE 1

| | | Additives | |
|---|---|---|---|
| Category | Code | % | |
| Antimicrobial | GP25-C00 | 0 | Inorganic biocide (zeolite) Sourced |
| Agents | GP25-C05 | 5 | from: Jishim Tech Co., Lid |
| | GP25-C10 | 10 | (Korea), CWT-A (brand name). |
| | GP25-C20 | 20 | |
| | GP25-C50 | 50 | |
| | GP25-B01 | 1 | BAC (benzalkonium chloride) Sourced |
| | GP25-B02 | 2 | from Mason Chemical, Company, IL, |
| | GP25-B05 | 5 | under NOBAC (brand name). |
| | GP25-B10 | 10 | |
| | GP25-D01 | 1 | DDAC (didecyl dimethyl ammonium |
| | GP25-D02 | 2 | chloride) Sourced from, Lonza Inc, NJ, |
| | GP25-D05 | 5 | BARDAC 2250 (brand name). |
| | GP25-D10 | 10 | |

Films containing the various amounts of the antimicrobial agents of Table 1 were made as follows. A twin-screw extruder (PRISM USALAB x16, available from Thermo Electric Co., Inc.) was used to make co-extruded film samples that contain an antimicrobial agent. The extruder specifications were as follows:
16 mm diameter screw
L/D=40 (L=640 mm)
10 heating zones+die
Maximum velocity=1000 rpm
Maximum pressure=100bar
Maximum torque=24 mN

The following extruder set-up was used to manufacture the experimental film:
Flat slit die width: 152.40 mm (6″)
Flat slit die height (controls film thickness): 0.127 mm (0.010″)

Each coextruded film included one of the active ingredients of Table 1.

The extruder feed zone was heated to 110° C., the following extruder zones 2-9 were heated to 125° C., and the die was heated to 130° C. The material was extruded.

### Dissolution Test “A”:

I. Preparation of Specimens:
   a. Cut 9 film specimens (approximately 0.75″×2.5″ or 0.07-0.12 g each). Record the mass of each specimen.
   b. Match each specimen with a tall 2 oz glass jar and lid. Fill each jar with enough buffered water so that the water is 100 times the weight of the film. Three jars are to be filled with a pH 5 buffered solution, three with a pH 7, and 3 with a pH 9 buffered solution.
      i. The buffered solutions contain:
         1. pH 5: 990 g tap water, 10 g sodium citrate, 1.89 g citric acid
         2. pH 7: 990 g tap water, 10 g sodium citrate, 0.18 g citric acid
         3. pH 9: 990 g tap water, 10 g sodium citrate, 1.02 g triethanolamine
   c. Heat one jar from each pH to 60° C., and one jar from each pH to 40° C. The last jar of each pH remains at room temperature (approx. 20° C.)
II. Testing of specimens:
   a. Gather the film specimens, a stopwatch, a glass stir-rod, and the jars of buffered water.
   b. Drop a film specimen into each jar, using the glass rod to submerge the film specimen if necessary. Do not drop the sample onto the wall of the jar, as the film will adhere and take longer to dissolve.
   c. Start the timer immediately after submerging the film specimen.
   d. Record the time that the film is 95%+dissolved. Swirl the jar if necessary to check to see if the film is dissolved. Some films cloud the water and make it difficult to discern when the specimen is dissolved.

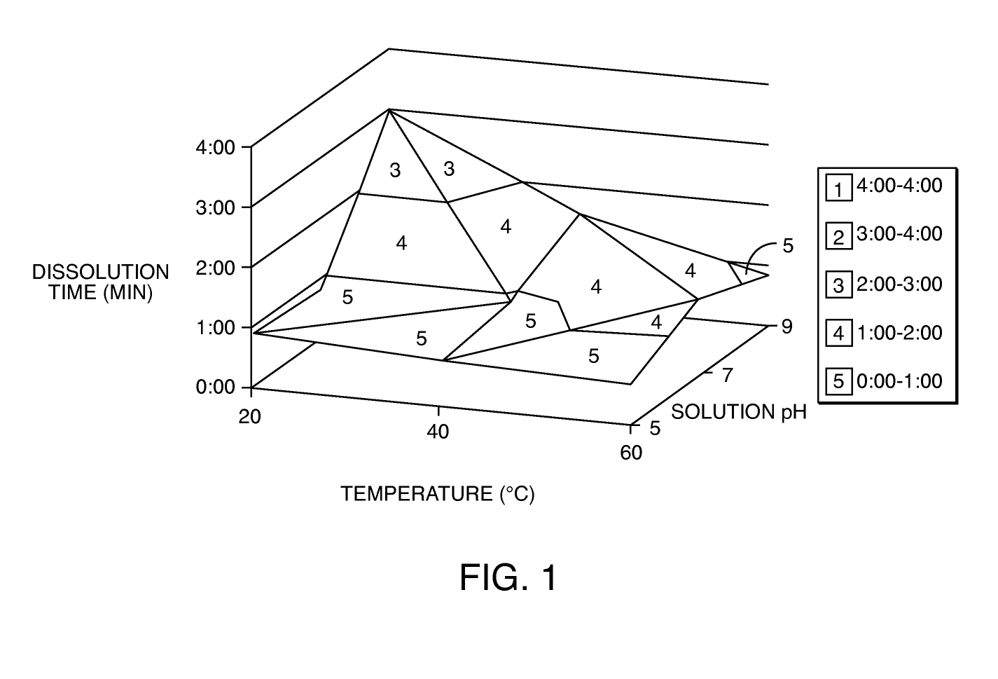

FIG. 1 is a three-dimensional graph showing the dissolution time for antimicrobial GP25-0O5, at varying pH and temperature. The longest dissolution time of three minutes is shown at a condition of pH 9 and 20° C. In contrast, the shortest dissolution time of less than 1 minute is shown at a condition of pH 5 and 60° C.

Zone of Inhibition Test: In this test method, the test material is brought into contact with a known population of microorganisms on an agar plate for a specified period of time. At the end of the contact time, the area of inhibited colony formation around the test material is measured. The size of this area of no growth is a measure of leaching of the antimicrobial agent from the test material.

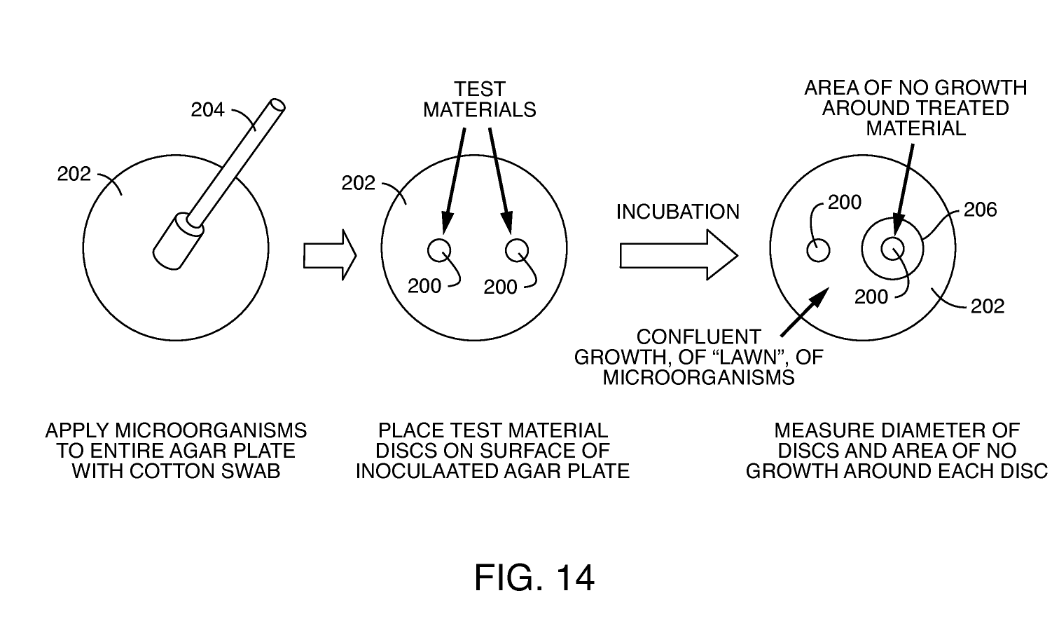

Referring to FIG. 14, the test material 200 is cut into small discs and placed on an agar plate 202 evenly spread with a test microorganism with a cotton swab 204. The plates are incubated for 24 hours at ideal growth conditions. Following incubation, the diameter of the circle of no growth 206 around the disc 200 is measured. The zone of inhibition is reported as the difference between the sample disc diameter and the average of the measured no growth zone diameters.

Materials and Reagents:
Microorganisms: frozen stock of Staphylococcus aureus (ATCC 27660) and Pseudomonas aeruginosa (ATCC 15442).
Mueller-Hinton agar (MHA) plates or equivalent plated media. Prepare following manufacturer's directions. Store at 4±2° C. Alternatively, pre-made plates can be utilized.
Mueller-Hinton broth (MHB) or equivalent liquid media. Prepare following manufacturer's directions. Store at 4±2° C. Alternately, pre-made media can be utilized.
   Sterile cotton swabs or equivalent.
Sterile forceps.
Positive control disc: Vanocymicin susceptibility discs (6 mm), 30 μg/disc (BD and Company; Sparks, Maryland).
Test material, cut into 8 mm discs.
Calipers or other measuring device.
Other ancillary lab supplies.

Supply Set-up:

1. Label growth media plates appropriately for testing codes.

2. Sterilize test material discs with UV exposure in Laminar flow hood for 15 minutes (both sides of disc), if required.

Inoculum:

1. Take appropriate measures to ensure culture purity.

2. Staphylococcus aureus or Pseudomonas aeruginosa is inoculated from an overnight plate or MHB into 5 ml of sterile MHB in a 35° C. incubator for 18-24 hrs.

3. The overnight culture is then adjusted using MHB to the 0.5 McFarland barium sulphate standards (1×108 CFU/ml) or approximately 0.15 OD with a 0.2 cm light path at 660 nm.

4. Discard the cell suspension if it is not used within 30 to 60 min after preparation. Zone of Inhibition Bioassay Procedure:

1. Pre-warm the MHA plates to room temperature. The number of plates required per strain will depend on the number of test materials to be tested and their anticipated zone inhibition diameters; discs should be placed on plates so that zones of inhibition do not overlap.

2. The surface of the plates should be dry. If not, dry the plates (with lids ajar) in a 35° C. incubator for 20-30 min just prior to inoculation. There should be no visible droplets of moisture on the surface of the agar or on the lids of the plates when they are inoculated.

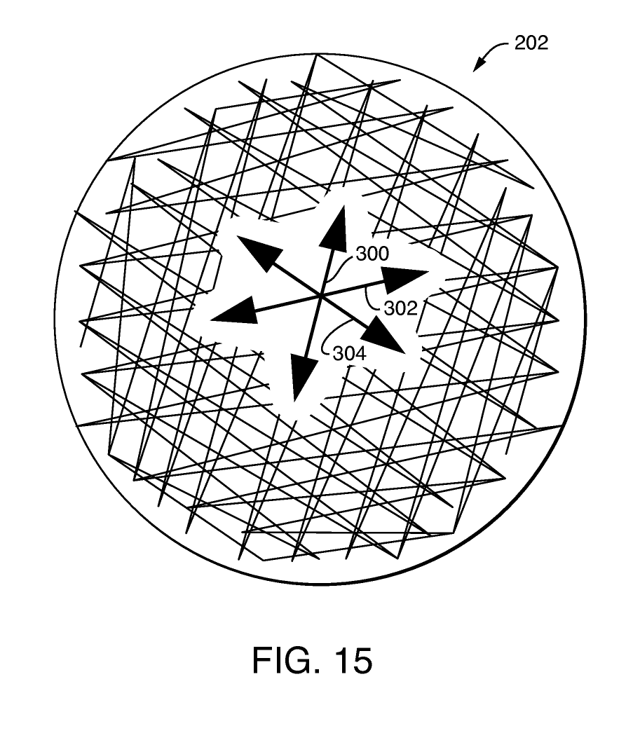

3. Moisten a sterile applicator swab in the standardized cell suspension and express any excess moisture by rotating the swab against the glass above the liquid in the tube. Referring to FIG. 15, inoculate the entire surface of each agar plate 202, inoculating the surface completely in three different directions 300, 302, 304 to ensure uniform growth.

(It is recommended that cotton swabs with wooden handles be used for this procedure. Swabs made of synthetic materials do not soak up sufficient suspension to inoculate the entire surface of the plate. Swabs with plastic handles bend when excess suspension is being expressed and may splatter liquid out of the tube.)

4. Repeat step 3 to inoculate additional plates as needed.

5. Store the inoculated plates at room temperature for 10-15 min to allow the medium to absorb the moisture from the inoculum.

6. Apply discs of test material to the surface of the inoculated medium with a sterile forceps and tap them to ensure that they are in complete contact with the agar surface. A positive control (vancomycin disc) and negative control (uncoated disc) should be used on each plate. All discs should be approximately the same distance from the edge of the plate and from each other (FIG. 15). In addition, all the discs should be positioned so the area of no growth that may develop around them to do not overlap.

7. Invert the inoculated plates and incubate them at 35° C. for 18-24 hours.

8. Examine the plates from the back, viewed against a black background and illuminated with reflected light. With calipers, measure the diameter of each zone of inhibition to the nearest whole millimeter.

Calculation: The zone of inhibition is equal to the diameter of the no-growth area minus the diameter of the disc.

The inhibition zone sizes given in this test protocol are derived from test methods used at the Center for Disease Control as well as AATCC Method 147-1998 (19) based on the National Committee for Clinical Laboratory Standards (20-21) and ASTM E2149-01 step 12.2 (22). The diameters of zones of inhibition may vary depending on many factors including medium base, humidity, and the age of the medium. Thus, it is important to follow one protocol as closely as possible to obtain results comparable between labs, personnel, and experiments. It may be necessary to determine zone interpretative sizes for disc diffusion results that are appropriate to local conditions. These criteria may be determined with use of reference strains and known challenge compounds and amounts.

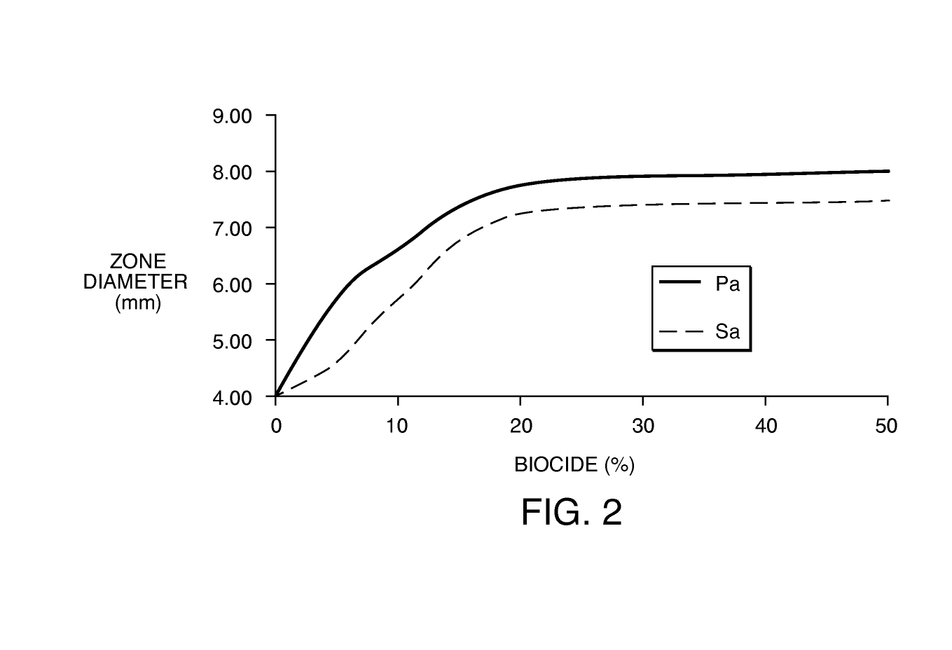

Results: FIG. 2. shows the result of the zone of inhibition testing for films containing antimicrobial agent CWT-A. This agent was more effective against Pseudomonas aeruginosa (Pa) than Staphylococcus aureus (Sa), but the effectiveness against both microbes plateaued when the film contained 20% or more of the antimicrobial agent.

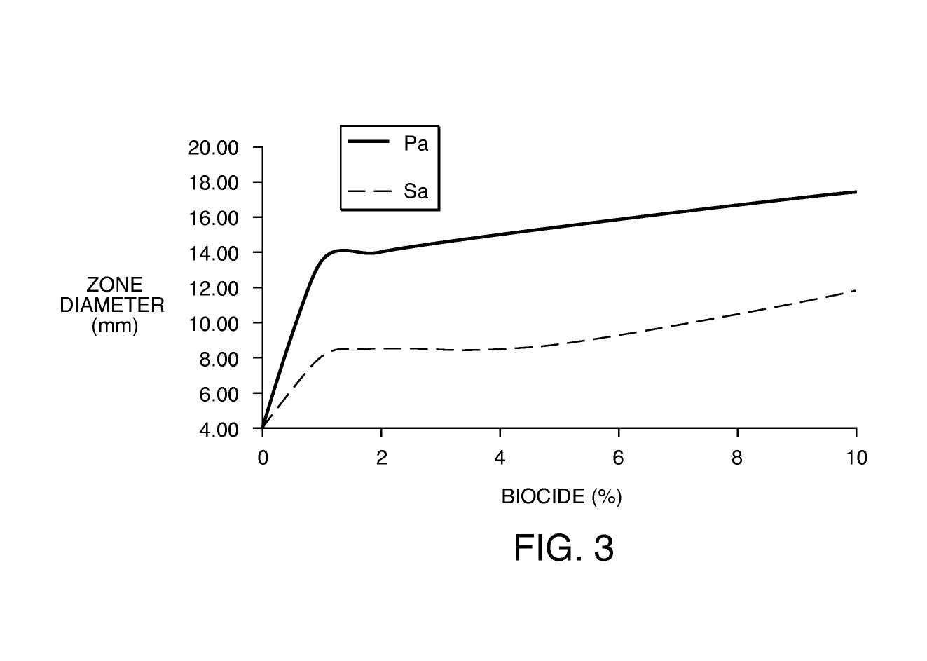

FIG. 3 shows the result of the zone of inhibition testing for films containing antimicrobial agent DDAC. This agent was significantly more effective against Staphylococcus aureus (Sa) than Pseudomonas aeruginosa (Pa). The effectiveness against Sa microbes went from a zone of 4 mm to 14 mm between 0% and about 1% DDAC. When the film contained from about 1% and 10% DDAC, the zone of inhibition of the Sa microbes went from about 14 mm to about 17 mm. The effectiveness against Pa microbes went from a zone of 4 mm to about 7 mm between 0% and about 1% DDAC. When the film contained from about 1% and 10% DDAC, the zone of inhibition of the Pa microbes went from about 9 mm to about 12 mm, plateauing at about 9 mm between about 1% and 4% DDAC.

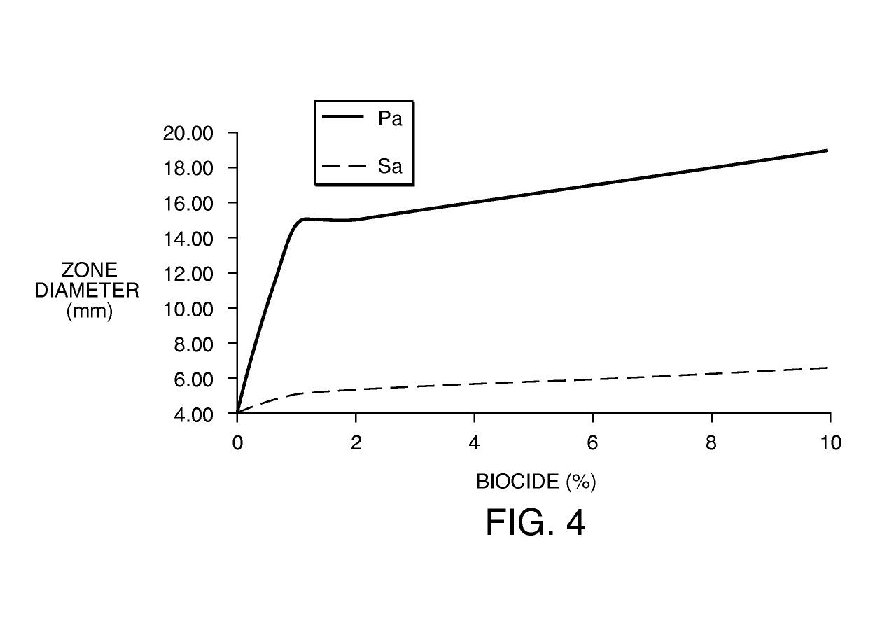

FIG. 4 shows the result of the zone of inhibition testing for films containing antimicrobial agent BAC. This agent was much more effective against Staphylococcus aureus (Sa) than Pseudomonas aeruginosa (Pa). The effectiveness against Sa microbes went from a zone of 4 mm to 14 mm between 0% and about 1% BAC. When the film contained from about 1% and 10% BAC, the zone of inhibition of the Sa microbes went from about 15 mm to about 19 mm. The effectiveness against the Pa microbes went from a zone of 4 mm to about 6mm between 0% and about 10% BAC.

Tensile Test: Prior to testing, samples were initially conditioned at 75° F/50% relative humidity for 24 hours. Thereafter, the strip tensile strength values were determined in accordance with ASTM Standard D-5034. A constant-rate-of-extension type of tensile tester was employed. The tensile testing system was a Synergie 200 tensile frame. The tensile tester was equipped with TESTWORKS 4.08B software from MTS Systems Corp. to support the testing. An appropriate load cell was selected so that the tested value fell within the range of 10-90% of the full scale load. The film samples were initially cut into dog-bone shapes with a center width of 3.0 mm before testing. The samples were held between grips having a front and back face measuring 25.4 millimeters×76 millimeters. The grip faces were rubberized, and the longer dimension of the grip was perpendicular to the direction of pull. The grip pressure was pneumatically maintained at a pressure of 40 pounds per square inch. The tensile test was run using a gauge length of 18.0 millimeters and a break sensitivity of 40%. Five samples were tested by applying the test load along the machine-direction and five samples were tested by applying the test load along the cross direction. During the test, samples were stretched at a crosshead speed of about 127 millimeters per minute until breakage occurred. The modulus of elasticity, peak load, peak stress, elongation (percent strain at break), and energy per volume at break (total area under the stress-strain curve) were measured. The tensile test results showed that the films have excellent mechanical properties for high-speed converting processes. This allows the films to easily be placed into articles such as the absorbent articles and laminates described herein.

### Test Results:

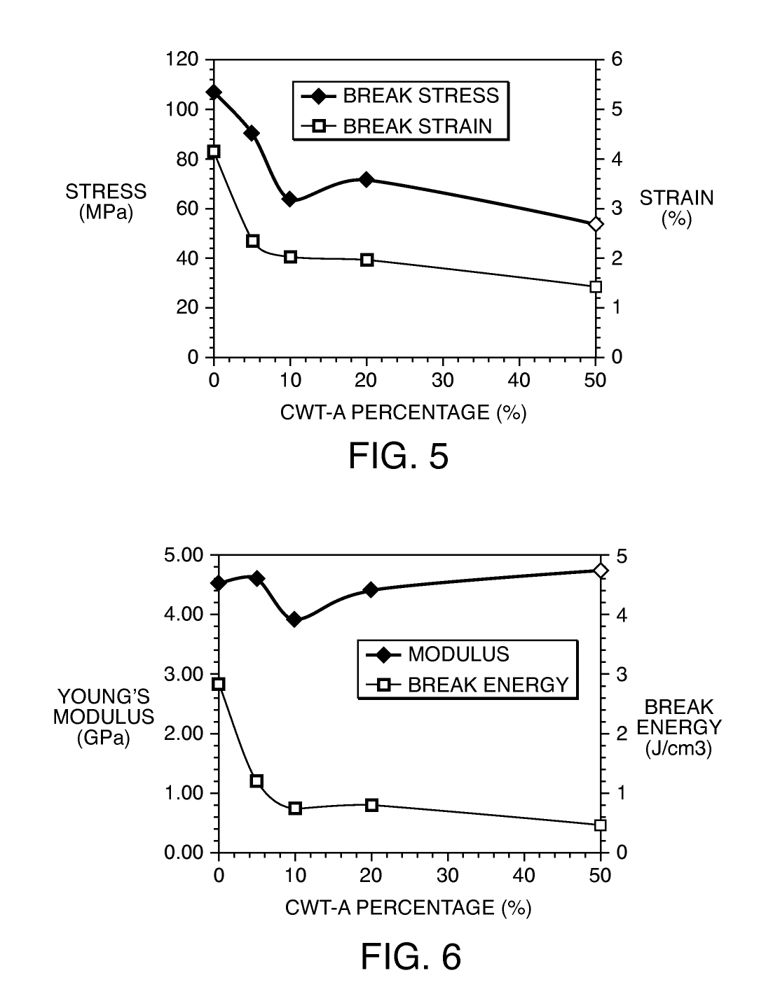

FIGS. 5-10 show the test results from the tensile tests described above.

Referring to FIG. 5, a dual chart shows how the stress and strain vary with the percentage of antimicrobial in the tested film, the antimicrobial being zeolite. The greatest break stress occurs when the film contains 0% zeolite by weight. The break stress drops rapidly as zeolite is added, and plateaus somewhat at about 10% zeolite content by weight. Like the break stress, the break strain is greatest when the film contains 0% zeolite by weight, and generally plateaus after about 7% zeolite by weight has been added.

Referring to FIG. 6, a dual chart shows how the elasticity and toughness vary with the percentage of antimicrobial in the tested film, the antimicrobial being zeolite. The greatest elasticity occurs when the film contains 50% zeolite by weight. The least amount of elasticity is seen at about 10% by weight zeolite. Like the break stress, toughness is greatest when the film contains 0% zeolite by weight, and generally plateaus after about 10% zeolite by weight has been added.

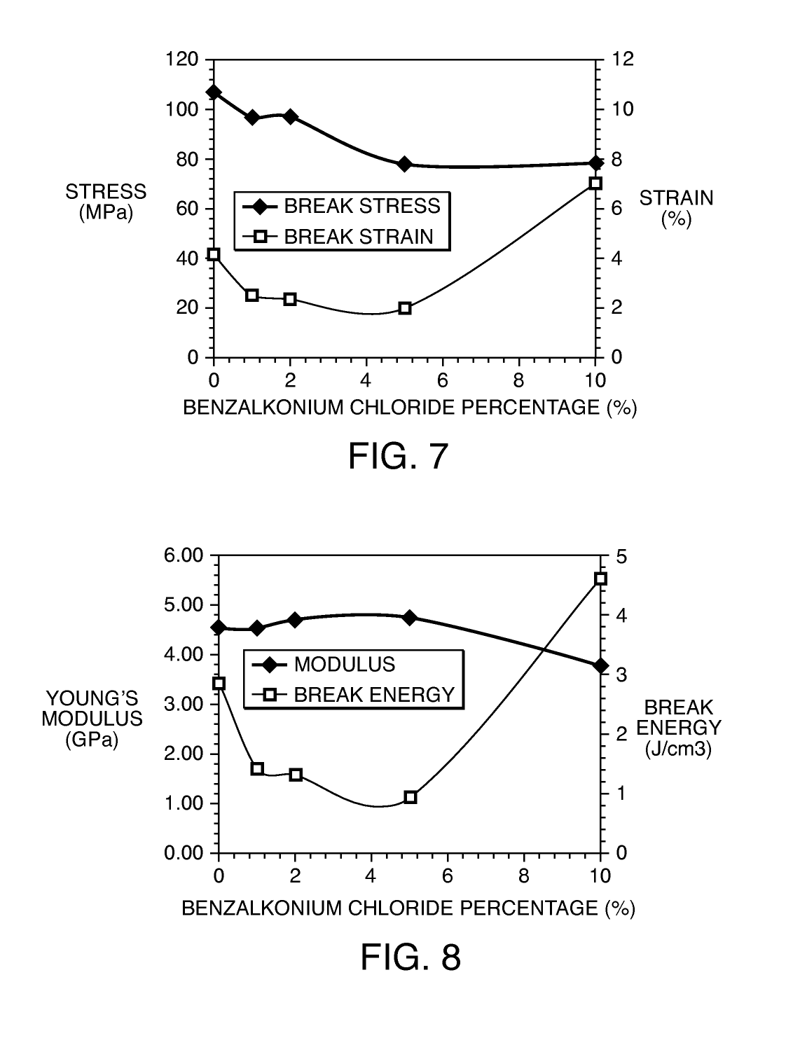

Referring to FIG. 7, a dual chart shows how the stress and strain vary with the percentage of antimicrobial in the tested film, the antimicrobial being benzalkonium chloride. The greatest break stress occurs when the film contains 0% benzalkonium chloride by weight. The break stress drops as benzalkonium chloride is added, and plateaus somewhat at about 5% benzalkonium chloride content by weight. Unlike the break stress, the break strain is greatest when the film contains 10% benzalkonium chloride by weight, and generally plateaus when between about 1% and 5% benzalkonium chloride by weight has been added.

Referring to FIG. 8, a dual chart shows how the elasticity and toughness vary with the percentage of antimicrobial in the tested film, the antimicrobial being benzalkonium chloride. The greatest elasticity occurs when the film contains 4% benzalkonium chloride by weight. The least amount of elasticity is seen at about 10% by weight benzalkonium chloride. Unlike the break stress, toughness is greatest when the film contains 10% benzalkonium chloride by weight has been added, a sharp rise from its lowest toughness that occurs at about 4% benzalkonium chloride by weight.

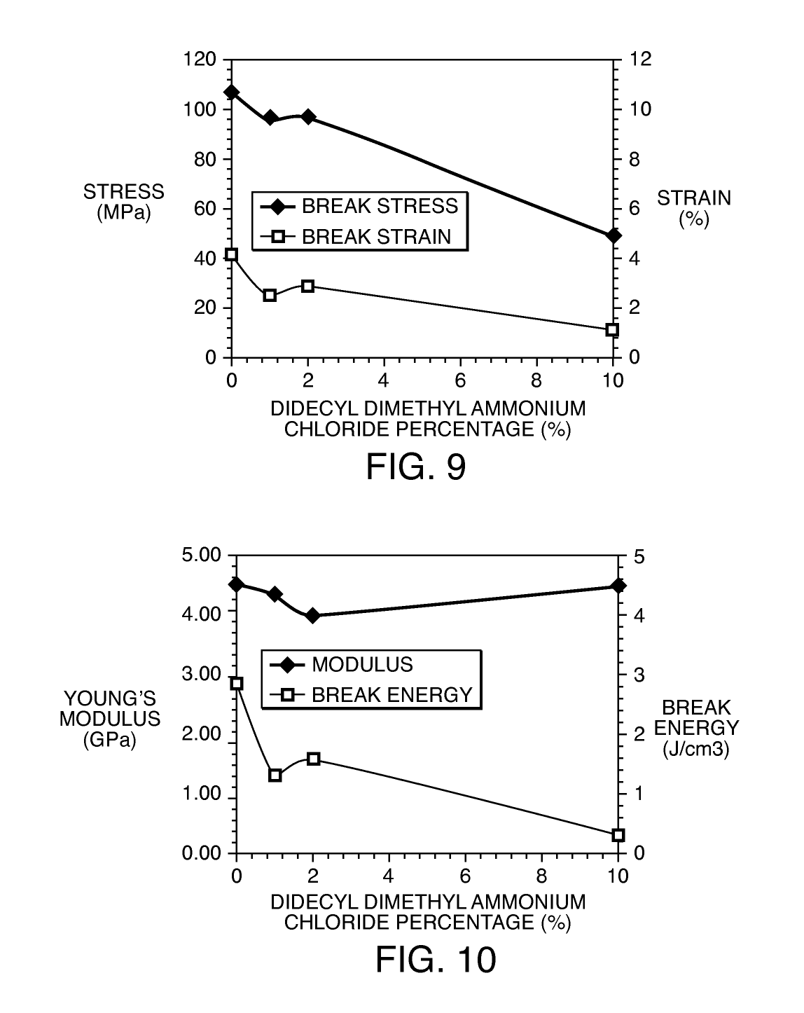

Referring to FIG. 9, a dual chart shows how the stress and strain vary with the percentage of antimicrobial in the tested film, the antimicrobial being didecyl dimethyl ammonium chloride. The greatest break stress occurs when the film contains 0% didecyl dimethyl ammonium chloride by weight. The break stress drops as didecyl dimethyl ammonium chloride is added, with only a small plateau between about 1 to 2% didecyl dimethyl ammonium chloride by weight. Like the break stress, the break strain is greatest when the film contains 0% didecyl dimethyl ammonium chloride by weight, and drops somewhat steadily as it is added.

Referring to FIG. 10, a dual chart shows how the elasticity and toughness vary with the percentage of antimicrobial in the tested film, the antimicrobial being didecyl dimethyl ammonium chloride. The greatest elasticity occurs when the film contains either 0% or 10% didecyl dimethyl ammonium chloride by weight. The least amount of elasticity is seen at about 2% by weight didecyl dimethyl ammonium chloride. Like the break stress, toughness is greatest when the film contains 0% didecyl dimethyl ammonium chloride by weight has been added. TH toughness drops steadily after 2% didecyl dimethyl ammonium chloride by weight has been added to the film, following a minor plateau between the 1% and 2% didecyl dimethyl ammonium chloride by weight has been added to the film.

### Odor Control System

The polymer described above may also be used to make an article capable of controlling odor in disposable absorbent articles. Generally, the antimicrobial active agents described supra may be completely replaced by or used in addition to an odor-control system having one or more odor-controlling active ingredients.

In one aspect of the disclosure, an odor-control article is an extruded water-soluble article made from a homogeneous water-soluble polymer matrix, (desirably an amorphous polyvinyl alcohol), having an extrusion temperature of 90° C. to 125° C., or in other aspects, 50° C. to 150° C., or 50° C. to 110° C. In one aspect there is mixed into the polymer matrix between 0.1% to 50% by weight of an odor-control system that includes one or more of the following odor-control agents: an enzyme inhibiter, a blocking agent, an antimicrobial agent, and an odor-absorption agent. The combined polymer matrix and odor-control system is mixed in an extruder as described above so that it becomes homogeneous. In another aspect, the extruded water-soluble article contains 1% to 30% by weight of the odor-control system incorporated into the polymer matrix.

As with the previous embodiments of the disclosure, other additives may be homogenously mixed with the polymer matrix and odor-control system. In one aspect of the disclosure, the resulting extruded article may include up to 50% thermoplastic starch by weight as described above (e.g. modified polysaccharide, GLUCOSOL 800, Chemstar Products Co.). In another aspect of the disclosure, the resulting extruded article includes up to 30% by weight of ethylene vinyl acetate, also described above (e.g. ESCORENE Ultra EVA, UL 8705, ExxonMobil Co.).

In one aspect, the extruded water-soluble article may take the form of a fiber-based nonwoven substrate. This type of substrate may be used as a liner or other disposable absorbent article component.

With respect to the odor-control system, various suitable materials may be used. Suitable enzyme inhibiters include Polyoxyethylene Glyceryl Monococoate (e.g. CETIOL HE, BASF Co., Port Arthur, Tex.), propanedioate, butanedioate, trans-butenedioate, silver, and copper. Suitable blocking agents include menthyl acetate (Sigma-Aldrich, St. Louis, Mo.). Suitable odor-absorption agents include activated carbon (NUCHAR WV-B 1500, MeadWestvaco Co.), silica (e.g. Snowtex 0, Nissan Chemical Co., Houston, Tex. Suitable antimicrobial agents include a silver-based zeolite (e.g. CWT-A, Jishim Tech Co., Ltd, Korea), and silver (e.g. SILVAGARD, Acrymed, Beaverton, OR). The following list of antimicrobial agents may be used in addition thereto: isothiazolone, alkyl dimethyl ammonium chloride, a triazine, 2-thiocyanomethylthio benzothiazol, methylene bis thiocyanate, acrolein, dodecylguanidine hydrochloride, a chlorophenol, a quaternary ammonium salt, gluteraldehyde, a dithiocarbamate, 2-mercatobenzothiazole, para-chloro-meta-xylenol, silver based compounds, chlorohexidine, polyhexamethylene biguanide, a n-halamine, triclosan, a phospholipid, an alpha hydroxyl acid, 2,2-dibromo-3-nitrilopropionamide, 2-bromo-2-nitro-1,3-propanediol, farnesol, iodine, bromine, hydrogen peroxide, chlorine dioxide, ozone, a botanical oil, a botanical extract, benzalkonium chloride, chlorine, sodium hypochlorite, or combinations thereof.

Film

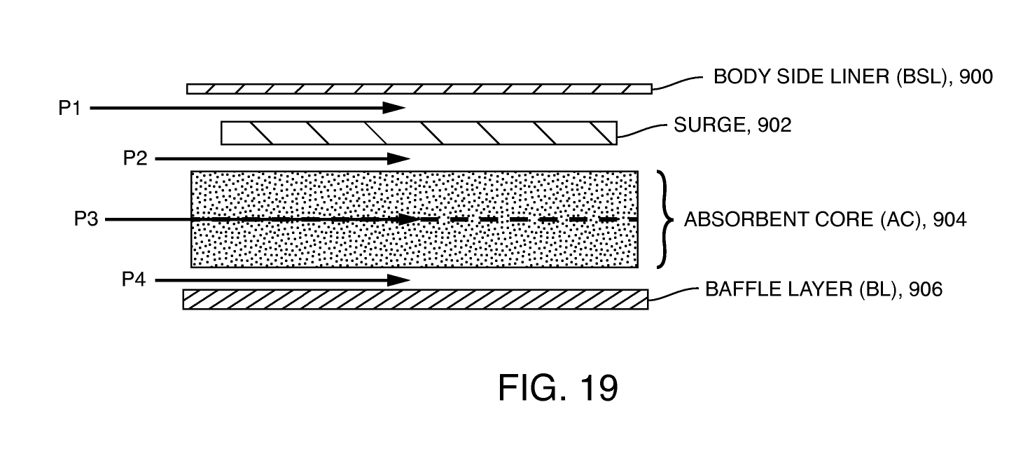

In one aspect of the disclosure, the extruded water-soluble article is a film which can be placed into a disposable absorbent article as shown in FIG. 19. The disposable absorbent article may be a bandage, a medical drape, a wipe, a towel, a sheet, a pad, a pant or a diaper.

There are several advantages of using a film in as a vehicle to deliver odor-control agents to the absorbent article, including but not limited to: 1) high loading capacity of the odor-control agent in the film; 2) no solvent is used, so no drying process is needed; 3) the ease of loading multiple odor-control agents into the film in one process; 4) the film can readily be laminated onto other substrates if desired; 5) the odor control system is in a polymer matrix so it does not evaporate; and 6) the active may be released over time as fluid migrates along the length of the film.

In one aspect of the disclosure, the extruded water-soluble film may have a water dissolution speed of 5 seconds to 30 minutes as determined by the Water Dissolution Test B disclosed herein.

In one aspect of the disclosure, the extruded water-soluble film may have a basis weight of 5 gsm to 500 gsm.

In other aspects of the disclosure, the extruded water-soluble article includes a skin-benefit agent selected from the group consisting of prebiotics, probiotics, humidity control material, skin pH-control material, a skin protectant that mitigates skin irritation caused by feces/urine, and combinations thereof.

Experimental Data for Film with Odor-Control System

A twin-screw extruder was used to melt/mix the water-soluble, amorphous polyvinyl alcohol polymer with the odor-control system at a temperature of 125° C. Each film was made by extruding the material through a flat die. Films containing single or multiple actives were made by this process, which may be referred to as “deodorant films”.

Table 2 shows the functional components of the odor-control system and the polymer components for each film tested. Each test is described below.

TABLE 2

| | | | | | | | | | Actives | | |
|---|---|---|---|---|---|---|---|---|---|---|---|
| | | | | | | | | | in | Film | |
| | Polymer Components | | | Functional Components | | | | | (1″ × 7″) | Basis | |
| Film | PVOH | EVA | Starch | AC | MTA | CWTA | CTL | Actives | strip | Wt | # of |
| Code # | (%) | (%) | (%) | (%) | (%) | (%) | (%) | (%) | (mg) | (gsm) | actives |
| 021813-4 | 50.0 | 12.5 | 31.2 | | | | 6.3 | 6.3 | 39.6 | 138.2 | 1 |
| 043013-1 | 70.6 | 17.6 | 0.0 | 11.8 | | | | 11.8 | 71.2 | 133.6 | 1 |
| 050613-4 | 54.5 | 13.6 | 20.7 | | | 11.2 | | 11.2 | 50.3 | 99.5 | 1 |
| 050613-6 | 47.8 | 11.9 | 35.8 | | 4.5 | | | 4.5 | 20.3 | 99.8 | 1 |
| 043013-3 | 62.2 | 15.5 | 0.0 | 10.4 | | | 11.9 | 22.3 | 132.4 | 131.4 | 2 |
| 050613-1 | 62.2 | 15.6 | 0.0 | 11.1 | | 11.1 | | 22.2 | 135.4 | 135.1 | 2 |
| 050613-5 | 53.6 | 13.4 | 18.2 | | | 9.8 | 5.0 | 14.8 | 202.6 | 303.2 | 2 |
| 050613-3 | 56.4 | 14.1 | 0.0 | 10.1 | | 10.1 | 9.3 | 29.5 | 285.7 | 214.5 | 3 |

Key:
AC = activated carbon;
MTA = menthyl acetate;
CWTA = silver-based zeolite;
CTL = polyoxyethylene glyceryl monococoate
DISSOLUTION TEST “B”: Dissolution Test B was performed with the films of Table 2 using test method ASTM D5226-96 and the following parameters. Three specimens were tested for each code.

1. Film Sample Preparation

a. The eight samples (see Table 2 codes) were cut into 2.54 cm×15.24 cm specimens using a die and press.

b. Three specimens were cut from each sample.
2. Experimental Set up and Procedure

a. Deionized water was placed in a 1 L glass beaker and heated to 37° C. in a hot water bath.

b. The total mass of each strip was measured and recorded using a standard balance.

c. The mass of polymer to mass of odor-control system ratio was 0.5%. The appropriate mass of odor-control system was calculated using the total mass.

d. After the odor-control system was to temperature, an appropriate mass of same was obtained using the balance and placed in a 250 mL glass beaker with a 1.5 inch magnetic stir rod.

e. The beaker was then placed on a VWR Hotplate/Stirrer with a feedback thermometer. The temperature of the medium was then checked to ensure the medium was at 37° C. The stir rate was set to 100 rpms.

f. The film was then folded in half (to prevent film adherence to the sides of the beaker) and placed on the surface of the water. At this point, the timer was started.

g. The film in solution was closely monitored to observe the dissolution process of the film and determine the end point.

h. The end point was determined primarily by the size of the intact film left in solution.
i. 90% dissolved film was set as the standard for the end point. When most of the large particles were dissolved the end point was set.
ii. The goal was to go from 6 in² to about 0.6 in² surface area. If the film curled into a ball, this translates to a radius of about 0.64 cm (0.25 inch).
i. Haziness of the water or saline solution was a secondary factor. Many of the films are off-white or black, and upon dissolution cause the solution to become cloudy.
3. Accuracy Check

Because it may be challenging to determine the end point, an accuracy check was performed. The film solution was monitored for an additional time equivalent to two times the length of the trial to ensure the film did not further dissolve. For example, if the end point for a film was determined to be 45 seconds then the film was monitored for an additional 90 seconds to ensure further dissolution did not occur. The accuracy check works well for rapidly dissolving films. However, performing the accuracy check on films that take a relatively long time to dissolve is not practical.

### Test Results:

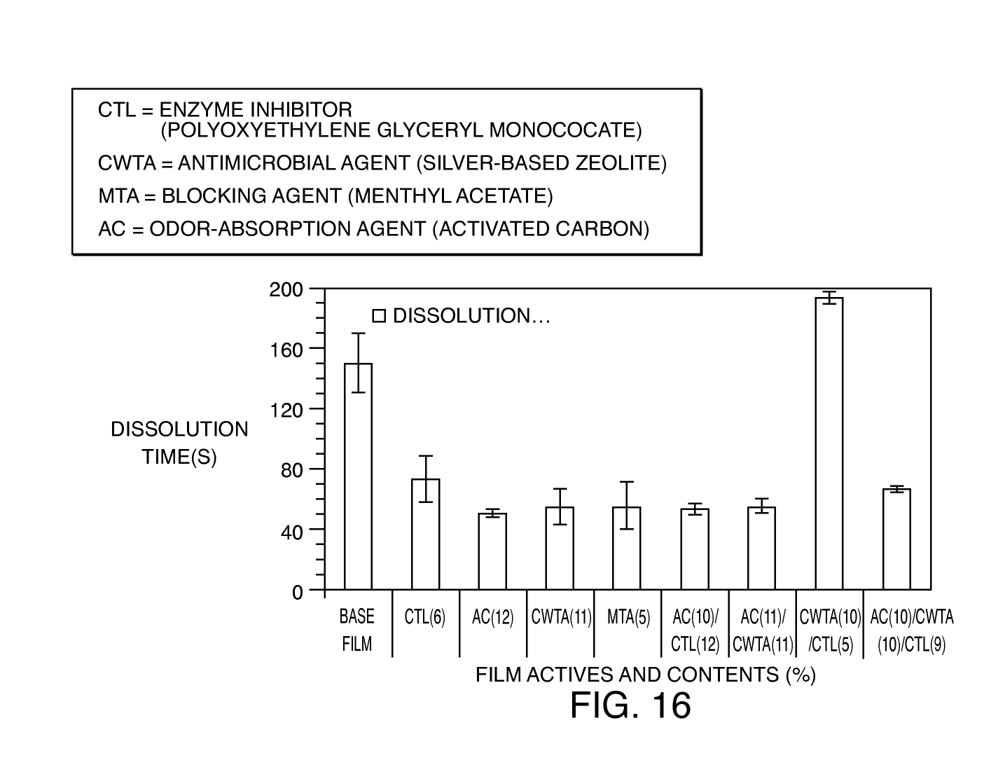

Referring now to FIG. 16, under the Dissolution Test B conditions, the deodorant films demonstrated a dissolution rate of 30 seconds to 3 minutes, with most films dissolving in about 40-60 seconds. The dissolution rate increased with the inclusion of a non-soluble polymer components such as EVA, which is one of mechanisms used to control the release rate of odor-control agents from the film matrix.

Tensile Test: Standard tensile test method ASTM D882-98 was used with the following parameters to perform the tests:

Specimen size: 101.6 mm×9.525 mm (4″×⅜″) strip

Grip separation: 5.08 cm (2 inches)

Test speed: 50.8 cm/min (20 inches/min)

Test input data: Specimen thickness in mm

Sample size: >8

Report: Stress, elongation, modulus, break energy

Specimen preparation: ASTM 6287

### Test Results:

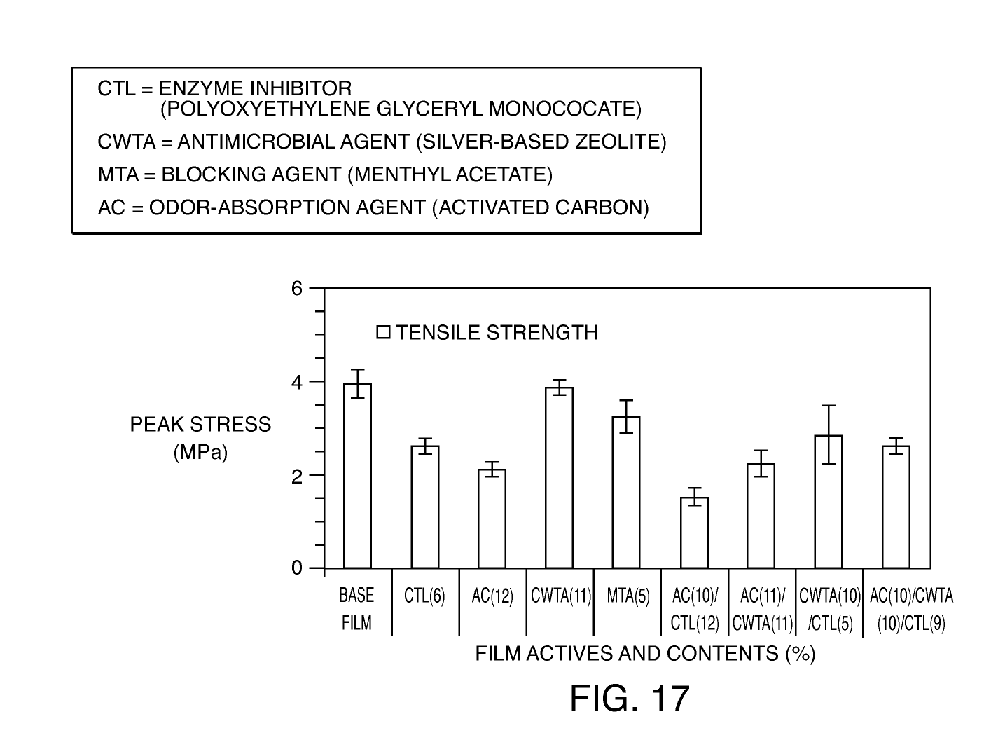

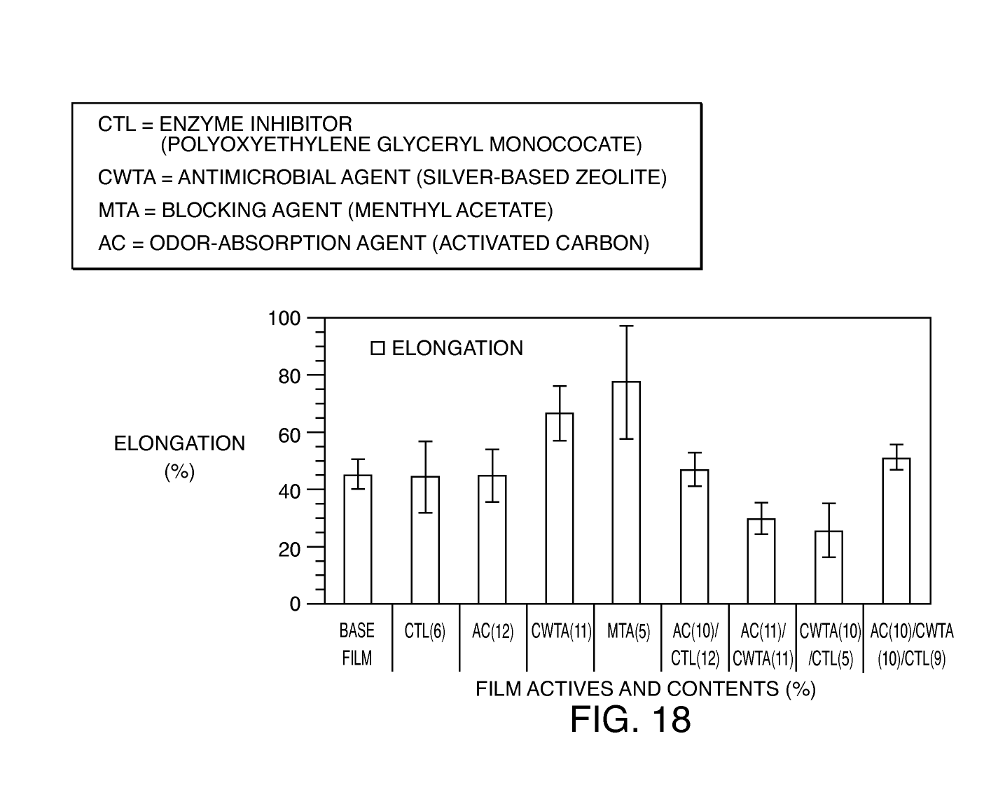

Referring to FIGS. 17 and 18, the tensile strength of the deodorant films ranged from about 1.5 to about 4 MPa, and the elongation ranged from about 20% to about 80%. Generally, the functional ingredients serve to weaken the polymer (as compared to Base Film). The deodorant film showing the greatest tensile strength was the film that contained silver-based zeolite. The deodorant film demonstrating the second best tensile strength was that containing menthyl acetate. The silver-based zeolite film demonstrated higher elongation than all but one other deodorant film, that containing menthyl acetate.

Fluid Intake Test: A Cradle Test Method was used for this test. The primary purpose of this test was to determine how deodorant films affect fluid intake. The secondary purpose of this test was to determine how fluid intake is affected by the location of the deodorant film within the absorbent article.

The absorbent articles tested were made by incorporating a 2.54 cm (1 inch) wide, 17.18 cm (7 inch) long strip of deodorant film into commercially-available: moderate-absorbency, long-length, POISE pads (Kimberly-Clark Corporation).

The cradle test for fluid intake of POISE pads with deodorant film was conducted as follows.
1. Sample was placed into a cradle.
2. The sample was insulted with with 37C saline at a fluid rate of 8 ml/minute. There was four insults: 30 ml insults with 15 minute intervals between insults; 120 ml fluid in total.
3. Measured was the length of time it took for the fluid to be completely absorbed by the sample.
4. Flowback is the amount of unabsorbed fluid after the third insult. More specifically, it is defined as the amount of fluid that can be absorbed from an insulted specimen onto a blotter as it is subjected to a predetermined vacuum pressure for a specified amount of time.

One 2.54 cm (1 inch) by 17.78 cm (7 inch) specimen was placed at one of four positions in the absorbent article (see, FIG. 19: P1=beneath the body side liner 900, P2=beneath the surge 902, P3=the middle of absorbent core 904, and P4=between the absorbent core 904 and baffle layer 906). The longitudinal axis of the film matched that of the pad as the film was centered on the pad surface as close as possible. See, Table 3 showing the components for each sample used in the fluid intake test.

TABLE 3

| | | | | | | | | | Actives | |
|---|---|---|---|---|---|---|---|---|---|---|
| | | | | | | | | | in | |
| | Polymer Components | | | Functional Components | | | | Active | (1″ × 7″) | Basis |
| Film | PVOH | EVA | Starch | AC | MTA | CWTA | CTL | Content | strip | Weight |
| Code # | (%) | (%) | (%) | (%) | (%) | (%) | (%) | (%) | (mg) | (gsm) |
| 073113-4 | 62.2 | 15.6 | 0.0 | 11.1 | | 11.1 | | 22.2 | 119.0 | 118.5 |
| 073113-5 | 70.0 | 17.5 | 0.0 | 6.3 | | 6.3 | | 12.5 | 79.2 | 140.4 |
| 073113-6 | 74.9 | 18.7 | 0.0 | 3.2 | | 3.2 | | 6.4 | 29.5 | 101.6 |
| 043013-3 | 62.2 | 15.5 | | 10.4 | | | 11.9 | 22.3 | 132.4 | 131.4 |
| 073113-9 | 75.3 | 18.8 | | 2.8 | | | 3.0 | 5.8 | 89.8 | 341.1 |
| 073113-1 | 53.6 | 13.4 | 18.1 | | | 9.8 | 5.1 | 14.9 | 193.9 | 288.4 |
| 073113-2 | 64.6 | 16.2 | 10.5 | | | 5.7 | 3.1 | 8.7 | 105.6 | 267.8 |
| 073113-10 | 69.0 | 17.2 | 9.2 | | | 2.3 | 2.3 | 4.6 | 33.5 | 162.0 |
| 081613-1 | 56.1 | 0.0 | 24.3 | | | 13.1 | 6.6 | 19.6 | 63.2 | 71.3 |
| 081613-2 | 56.1 | 0.0 | 24.3 | | | 13.1 | 6.6 | 19.6 | 49.6 | 55.9 |

Test Results:

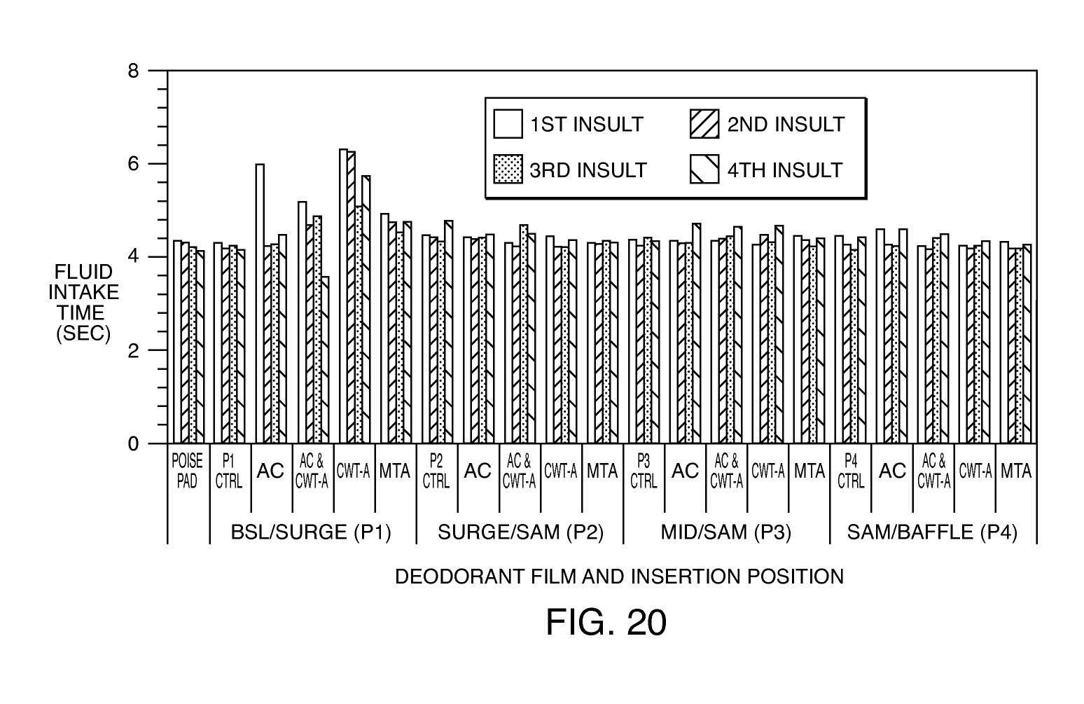

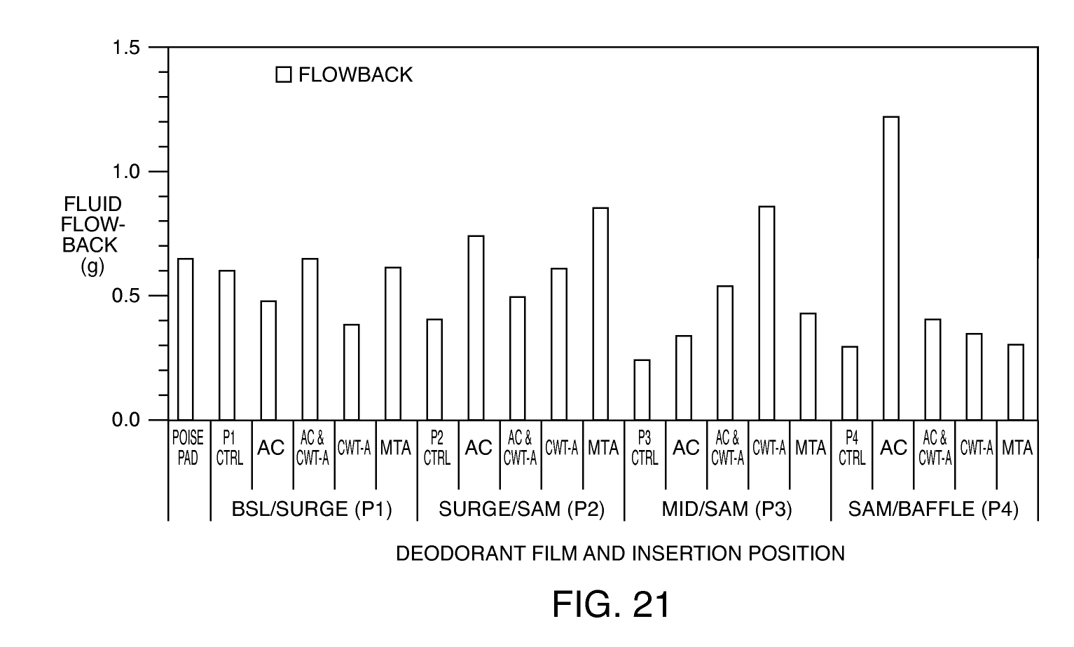

The fluid intake test demonstrated that the incorporation of a deodorant film strip into a POISE pad at varied interface positions did not produce significant impact on the fluid intake time. See, FIGS. 19 and 20. Likewise, the fluid flow-back was not significantly affected with the exception of the placement of activated carbon film between the absorbent core 904 and baffle 906. See FIGS. 19 and 21.

The deodorant film that remains undissolved after the Cradle Test has been performed is referred to as “leftover.”

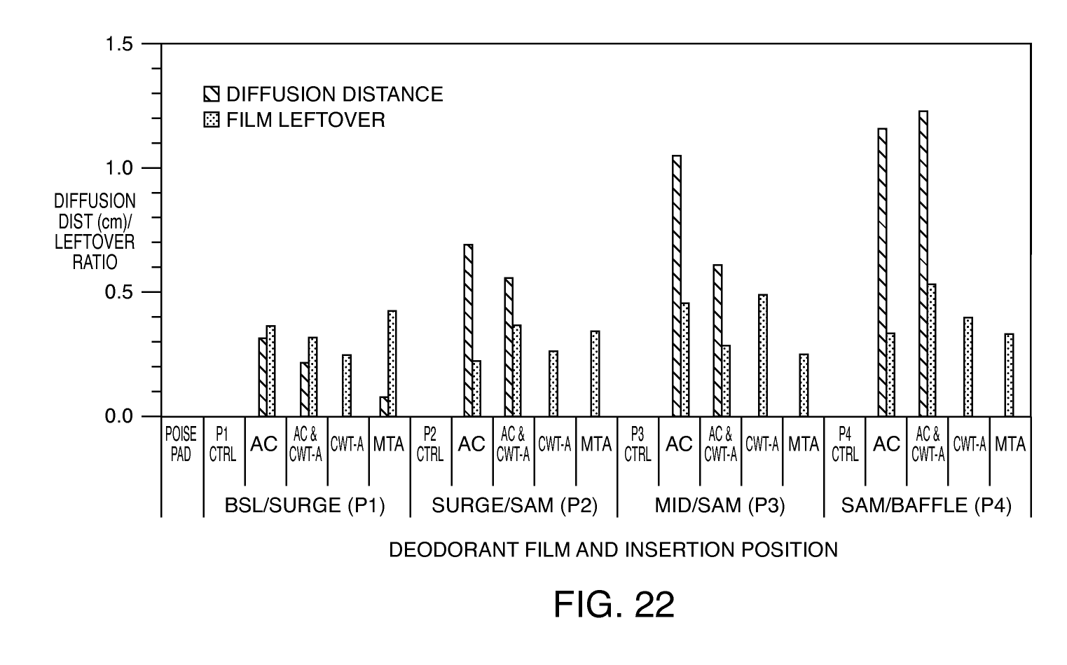

The “diffusion distance” refers to the distance that solid active particles (e.g. activated carbon) are able to travel away from the original film position with fluid flow. Referring to FIG. 22, the activated carbon particle in the film embedded beneath the body side liner, i.e., P1 position, travelled 1-3 mm away from its original position; while the particles at P4 position travelled more than 1 cm. This aids in distributing the absorbent particles over a larger area, increasing efficiency.

Urine Odor Ranking Panel: The odor reduction efficacy of the deodorant film was evaluated with a human urine odor-ranking panel (ORP) study as follows.
1. Referring to FIG. 19, a 2.54 cm by 17.78 cm film strip was inserted at location P2 between the surge 902 and absorbent core 904 of a Moderate-Absorbency/Long-Length POISE pad.
2 Test urine fluid was prepared by following standards and criteria:
   a) collected urine was pooled, filtered and sterilized;
   b) sterilized urine was inoculated with log 6 of four different bacteria: e-coli, enterococcue faecalis, proteus mirabilis, and klebsilla pneumonia
3. 26 ml of test urine was introduced to the product in an incubation jar. The jar was capped and stored at 37+/−2 C for 4 hours. Each jar was blind-coded by assigning a random three-digit number according to the study randomization.
4. A total of 12 panelists was used. Each panelist ranked a set of up to four products for Total Odor Intensity (all odor combined) and Urine Odor Intensity (any combination of sweaty, fishy, ammonia, and sulfur odors). The odor panelists ranked test samples from most odor (rank=1) to least odor (rank=4) for total overall odor intensity and for total urine intensity. Panelists also described the character of the odors in the sample.
5. Data was collected using paper ballots and electronically entered into the data base system for analysis. A proportional hazards model was used to analyze the ranking data. Generate was a log odds value for each code in the study.

### Test Results:

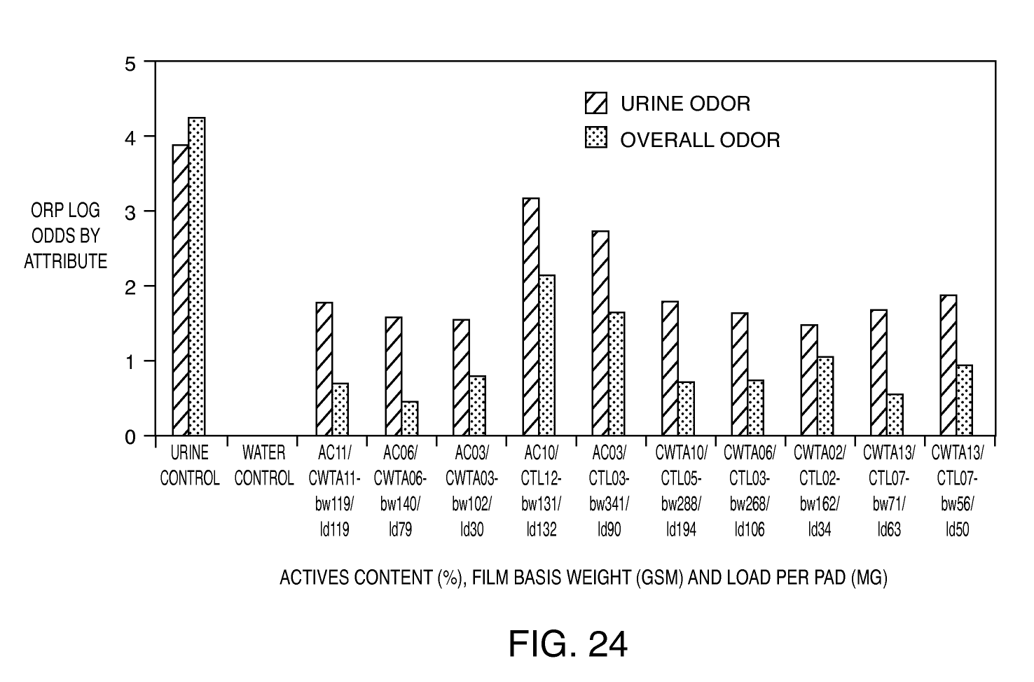

Referring now to TABLE 4 and corresponding FIG. 24, the first ORP study demonstrated that deodorant films containing a single odor-control agent can reduce urine odors by 1 to 2 log odd value. Of the odor control systems tested, the CWTA performed best. This is compared to a urine control which has log odd value of 4 to 4.5, and a water control log odd value near zero. Deodorant films containing two or three odor-control agents can reduce urine odors by up to 3 log odd value. There was very little difference between the various combinations of AC/CWTA, CTL/CWTA, and AC/CTL/CWTA. The study demonstrated that a combination of multiple odor-control agents synergistically reduce different types of odors.

TABLE 4

| | | | Film |
|---|---|---|---|
| | Active | Actives in (1″ × 7″) | Basis |
| | Content | strip | Weight |
| Film Code Name | (%) | (mg) | (gsm) |
| Urine Control | | | |
| Water Control | | | |
| AC11/CWTA11 - bw119/ld119 | 22.2 | 119.0 | 118.5 |
| AC06/CWTA06 - bw140/ld79 | 12.5 | 79.2 | 140.4 |
| AC03/CWTA03 - bw102/ld30 | 6.4 | 29.5 | 101.6 |
| AC10/CTL12 - bw131/ld132 | 22.3 | 132.4 | 131.4 |
| AC03/CTL03 - bw341/ld90 | 5.8 | 89.8 | 341.1 |
| CWTA10/CTL05 - bw288/ld194 | 14.9 | 193.9 | 288.4 |
| CWTA06/CTL03 - bw268/ld106 | 8.7 | 105.6 | 267.8 |
| CWTA02/CTL02 - bw162/ld34 | 4.6 | 33.5 | 162.0 |
| CWTA13/CTL07 - bw71/ld63 | 19.6 | 63.2 | 71.3 |
| CWTA13/CTL07 - bw56/ld50 | 19.6 | 49.6 | 55.9 |

Key:
AC = activated carbon
CTL = polyoxyethylene glyceryl monococoate
CWTA = silver-based zeolite
MTA = menthyl acetate
ACxx = xx % AC add-on
bwxxx = xxx gsm basis weight
ldxx = xx mg active per pad

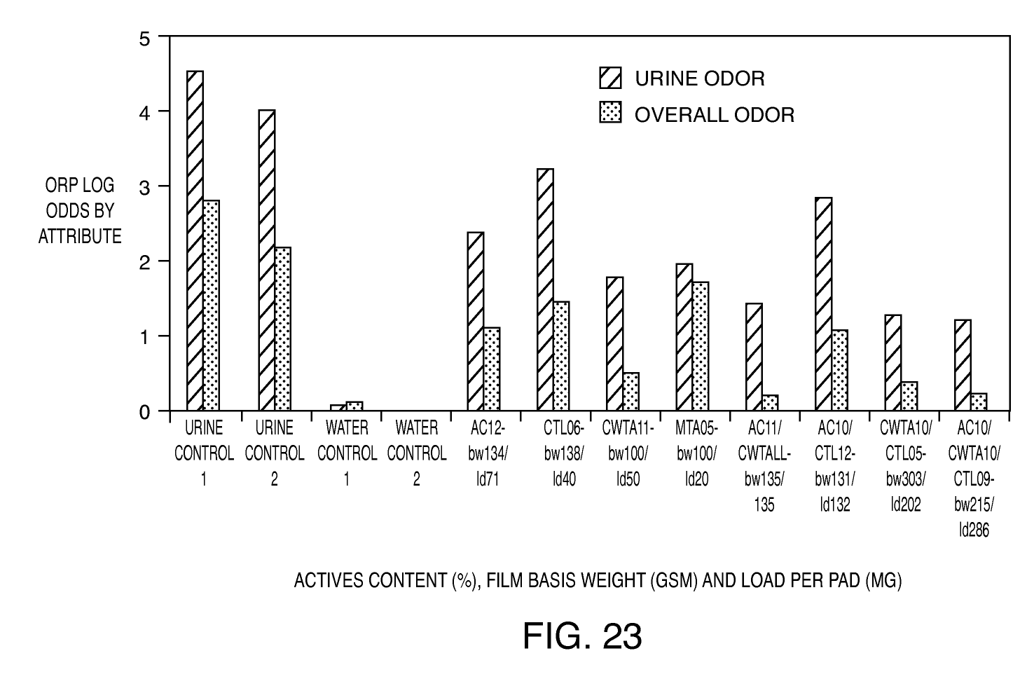

A second odor ranking panel was performed, and the data is portrayed below in TABLE 5 and in FIG. 23. The second ORP studied different combinations of odor-control agents to determine desirable loading and basis weights. The combination of CWTA and AC performed generally the same as a combination of CWTA and CTL.

TABLE 5

| | | Actives in | Film | |
|---|---|---|---|---|
| | Active | (1″ × | Basis | |
| | Content | 7″) strip | Weight | # of |
| Film Code Name | (%) | (mg) | (gsm) | actives |
| CTL06 - bw138/ld40 | 6.3 | 39.6 | 138.2 | 1 |
| AC12 - bw134/ld71 | 11.8 | 71.2 | 133.6 | 1 |
| CWTA11 - bw100/ld50 | 11.2 | 50.3 | 99.5 | 1 |
| MTA05 - bw100/ld20 | 4.5 | 20.3 | 99.8 | 1 |
| AC10/CTL12 - bw131/ld132 | 22.3 | 132.4 | 131.4 | 2 |
| AC11/CWTA11 - bw135/135 | 22.2 | 135.4 | 135.1 | 2 |
| CWTA10/CTL05 - bw303/ld202 | 14.8 | 202.6 | 303.2 | 2 |
| AC10/CWTA10/CTL09 - | 29.5 | 285.7 | 214.5 | 3 |
| bw215/ld286 | | | | |

Use same Key as Table 4

While the present invention has been described with reference to particular embodiments, which have been set forth in considerable detail for the purposes of making a complete disclosure, such embodiments are merely exemplary and are not intended to be limiting or represent an exhaustive enumeration of all aspects of the disclosure. It will be apparent that to those of skill in the art that numerous changes may be made in such details without departing from the spirit and principles of the disclosure.

1. An extruded water-soluble article comprising:
a homogeneous material comprising a water-soluble, amorphous polyvinyl alcohol matrix having an extrusion temperature of 90° C. to 125° C.; and
between 0.1% to 50% by weight of an odor-control system selected from the group consisting of an enzyme inhibiter, a blocking agent, an antimicrobial agent, an odor-absorption agent, and a combination thereof.

2. An extruded water-soluble article comprising:
a homogeneous material comprising a water-soluble, polymer having an extrusion temperature of 50 to 150° C.; and
between 0.1% to 50% by weight of an odor-control system comprising silver-based zeolite and polyoxyethylene glyceryl monococoate.

3. The extruded water-soluble article of claims 1 and 2 claim 1, wherein there is 1% to 30% by weight of the odor-control system.

4. The extruded water-soluble article of claim 1 further comprising up to 50% thermoplastic starch by weight.

5. The extruded water-soluble article of claim 1 further comprising up to 30% by weight of ethylene vinyl acetate.

6. The extruded water-soluble article of claim 1 wherein the article is a film.

7. The extruded water-soluble article of claim 6, wherein the film has a water dissolution speed from 5 seconds to 30 minutes as determined by a Water Dissolution Test of the present disclosure.

8. The extruded water-soluble article of claim 6, wherein the film has a basis weight of 5 gsm to 500 gsm.

9. The extruded water-soluble article of claim 1 further comprising a skin-benefit agent selected from the group consisting of prebiotics, probiotics, humidity control material, skin pH-control material, a skin protectant that mitigates skin irritation caused by feces/urine, and combinations thereof.

10. The extruded water-soluble article of claim 1, wherein the article is a fiber-based nonwoven substrate.

11. The extruded water-soluble article of claim 1, wherein the enzyme inhibiter comprises polyoxyethylene glyceryl monococoate.

12. The extruded water-soluble article of claim 1, wherein the blocking agent comprises menthyl acetate.

13. The extruded water-soluble article of claim 1, wherein the odor-absorption agent comprises activated carbon.

14. The extruded water-soluble article of claim 1, wherein the antimicrobial agent comprises a silver-based zeolite.

15. The extruded water-soluble article of claim 1, wherein the antimicrobial agent is selected from the group consisting of isothiazolone, alkyl dimethyl ammonium chloride, a triazine, 2-thiocyanomethylthio benzothiazol, methylene bis thiocyanate, acrolein, dodecylguanidine hydrochloride, a chlorophenol, a quaternary ammonium salt, gluteraldehyde, a dithiocarbamate, 2-mercatobenzothiazole, para-chloro-meta-xylenol, silver based compounds, chlorohexidine, polyhexamthylene biguanide, a n-halamine, triclosan, a phospholipid, an alpha hydroxyl acid, 2,2-dibromo-3-nitrilopropionamide, 2-bromo-2-nitro-1,3-propanediol, farnesol, iodine, bromine, hydrogen peroxide, chlorine dioxide, ozone, a botanical oil, a botanical extract, benzalkonium chloride, chlorine, sodium hypochlorite, and combinations thereof.

16. A personal absorbent article comprising: an absorbent member disposed between a water-impermeable backsheet and a water-permeable liner, wherein the liner has a body-facing surface and an opposite garment-facing surface; and the extruded water-soluble article of claim 1 attached to the liner or a surface of the absorbent member adjacent the liner.

17. An absorbent member having the film of claim 6 attached thereto, wherein the absorbent member is a bandage, a medical drape, a wipe, a towel, a sheet, a pad, a pant or a diaper.

18. An absorbent member comprising the film of claim 6, and an absorbent member disposed between a backsheet and a liner; wherein the film is disposed between the liner and the absorbent member or on top of the liner.

19. The extruded water-soluble article of claims 1 and 2 claim 1 further comprising activated carbon.

20. The extruded water-soluble article of claim 2, wherein there is 1% to 30% by weight of the odor-control system.

21. The extruded water-soluble article of claim 2 further comprising up to 50% thermoplastic starch by weight.

22. The extruded water-soluble article of claim 2 further comprising up to 30% by weight of ethylene vinyl acetate.

23. The extruded water-soluble article of claim 2 further comprising activated carbon.
